(19) United States
(12) Patent Application Publication
(10) Pub. No.: US 2005/0238701 A1
(43) Pub. Date: Oct. 27, 2005

(54) Fibrous structures comprising a transferable agent

(76) Inventor: Joerg Kleinwaechter, Hofheim Am Tanus (DE)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/105,997

(22) Filed: Apr. 14, 2005

Related U.S. Application Data
(60) Provisional application No. 60/658,686, filed on Mar. 4, 2005.
(60) Provisional application No. 60/565,104, filed on Apr. 23, 2004.
(60) Provisional application No. 60/565,105, filed on Apr. 23, 2004.

(51) Int. Cl. A61K 9/70

(52) U.S. Cl. 424/443

(57) ABSTRACT

Fibrous structures comprising a transferable agent, single- or multi-ply sanitary tissue products made therefrom and processes for making same are provided.

### CROSS REFERENCE TO RELATED APPLICATIONS

This application claims the benefit of U.S. Provisional Application Ser. No. 60/658,686 filed on Mar. 4, 2005 and U.S. Provisional Application Ser. No. 60/565,104 filed on Apr. 23, 2004 and U.S. Provisional Application Ser. No. 60/565,105 filed on Apr. 23, 2004.

### FIELD OF THE INVENTION

The present invention relates to fibrous structures comprising a transferable agent, single- or multi-ply sanitary tissue products made therefrom and processes for making same. More particularly, the present invention relates to fibrous structures comprising a user contacting surface comprising a transferable agent wherein the transferable agent is associated (present directly or indirectly on a surface of a fibrous structure and/or present within a fibrous structure) with a fibrous structure such that greater than about 6% by weight of the transferable agent is transferred from the fibrous structure to a surface during use (for example, contacting with the fibrous structure) by a user.

### BACKGROUND OF THE INVENTION

The common cold and allergies with their associated weeping eyes and runny noses are a bane to mankind. In addition to the difficulties in breathing, seeing, talking, and disposing of nasal discharge, an individual afflicted with these disorders frequently must contend with a nose and areas surrounding it which are sore and irritated and which are, frequently, red and inflamed thereby calling the attention of others to his plight. The irritation and inflammation-the redness-can have several causes. A prime one is, of course, the sheer necessity of frequently blowing the nose into a tissue or cloth and wiping nasal discharge from the nose and the area surrounding it. The degree of irritation and inflammation caused by blowing and wiping is strongly related to the surface roughness of the implement used. The degree of irritation and inflammation is also strongly related strongly related to the number of times the nose and its surrounding areas must be contacted with an implement; the use of an implement which is relatively weak or relatively nonabsorbent will require a greater number of contacts with the face weak or relatively nonabsorbent will require a greater number of contacts with the face than will the use of a stronger or more absorbent implement which is able to contain a greater quantity of nasal discharge.

There have been numerous previous attempts to correct the problem of irritation and inflammation caused by blowing and wiping. Examples of such previous attempts include adding lotions and/or beneficial skin chemistries to fibrous structures. However, it has been found that such previous attempts have failed to transfer sufficient amounts, such as a clinically beneficial amounts, and/or efficiently transfer such lotions and/or chemistries to a user's skin in order to provide clinical benefits.

Accordingly, a need exists for fibrous structures that comprise a transferable agent that is associated with the fibrous structure such that the transferable agent can be sufficiently transferred and/or efficiently transferred to provide a desired benefit, such as improving the condition of a user's irritated skin.

### SUMMARY OF THE INVENTION

The present invention fulfills the needs described above by providing a fibrous structure comprising a transferable agent that can be sufficiently and/or efficiently transferred to an opposing surface to provide a desired benefit.

In one example of the present invention, a fibrous structure comprising a transferable agent, wherein the transferable agent is associated with the fibrous structure such that greater than about 6% and/or greater than about 7% and/or greater than about 9% and/or greater than about 11% and/or greater than about 15% and/or greater than about 17% by weight of the transferable agent is transferred from the fibrous structure to a surface during use by a user, is provided.

In even another example of the present invention, a single- or multi-ply sanitary tissue product comprising a fibrous structure according to the present invention, is provided.

In even yet another example of the present invention, a process for treating a fibrous structure comprising the step of associating a transferable agent to a fibrous structure in need of treatment such that greater than about 6% by weight of the transferable agent is transferred from the fibrous structure to a surface during use by a user.

Accordingly, the present invention provides fibrous structures comprising a transferable agent, products made therefrom and processes for making same.

### BRIEF DESCRIPTION OF THE DRAWINGS

FIG. 1 is a schematic representation of a fibrous structure in accordance with the present invention;

FIG. 2 is a cross-sectional view of FIG. 1 taken along line 2-2;

FIG. 3 is a cross-sectional view of another example of a fibrous structure in accordance with the present invention;

FIG. 4 is a cross-sectional view of another example of a fibrous structure in accordance with the present invention;

FIG. 5 is a cross-sectional view of another example of a fibrous structure in accordance with the present invention;

FIG. 6 is a schematic representation of another example of a fibrous structure in accordance with the present invention.

### DETAILED DESCRIPTION OF THE INVENTION

Definitions

“Fiber” as used herein means an elongate particulate having an apparent length greatly exceeding its apparent diameter, i.e. a length to diameter ratio of at least about 10. Fibers having a non-circular cross-section are common; the “diameter” in this case may be considered to be the diameter of a circle having cross-sectional area equal to the cross-sectional area of the fiber. More specifically, as used herein, “fiber” refers to papermaking fibers. The present invention contemplates the use of a variety of papermaking fibers, such as, for example, natural fibers or synthetic fibers, or any other suitable fibers, and any combination thereof.

Natural papermaking fibers useful in the present invention include animal fibers, mineral fibers, plant fibers and mixtures thereof. Animal fibers may, for example, be selected from the group consisting of: wool, silk and mixtures thereof. Plant fibers may, for example, be derived from a plant selected from the group consisting of: wood, cotton, cotton linters, flax, sisal, abaca, hemp, hesperaloe, jute, bamboo, bagasse, kudzu, corn, sorghum, gourd, agave, loofah and mixtures thereof.

Wood fibers; often referred to as wood pulps include chemical pulps, such as kraft (sulfate) and sulfite pulps, as well as mechanical and semi-chemical pulps including, for example, groundwood, thermomechanical pulp, chemi-mechanical pulp (CMP), chemi-thermomechanical pulp (CTMP), neutral semi-chemical sulfite pulp (NSCS). Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified and/or layered web. U.S. Pat. No. 4,300,981 and U.S. Pat. No. 3,994,771 are incorporated herein by reference for the purpose of disclosing layering of hardwood and softwood fibers. Also applicable to the present invention are fibers derived from recycled paper, which may contain any or all of the above categories as well as other non-fibrous materials such as fillers and adhesives used to facilitate the original papermaking.

The wood pulp fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. Softwood fibers derived from the kraft process and originating from more-northern climates may be preferred. These are often referred to as northern softwood kraft (NSK) pulps.

Synthetic fibers may be selected from the group consisting of: wet spun fibers, dry spun fibers, melt spun (including melt blown) fibers, synthetic pulp fibers and mixtures thereof. Synthetic fibers may, for example, be comprised of cellulose (often referred to as “rayon”); cellulose derivatives such as esters, ether, or nitrous derivatives; polyolefins (including polyethylene and polypropylene); polyesters (including polyethylene terephthalate); polyamides (often referred to as “nylon”); acrylics; non-cellulosic polymeric carbohydrates (such as starch, chitin and chitin derivatives such as chitosan); and mixtures thereof.

“Fibrous structure” as used herein means a structure that comprises one or more fibers. Nonlimiting examples of processes for making fibrous structures include known wet-laid papermaking processes and air-laid papermaking processes. Such processes typically include steps of preparing a fiber composition, oftentimes referred to as a fiber slurry in wet-laid processes, either wet or dry, and then depositing a plurality of fibers onto a forming wire or belt such that an embryonic fibrous structure is formed, drying and/or bonding the fibers together such that a fibrous structure is formed, and/or further processing the fibrous structure such that a finished fibrous structure is formed. For example, in typical papermaking processes, the finished fibrous structure is the fibrous structure that is wound on the reel at the end of papermaking, but before converting thereof into a sanitary tissue product.

“Sanitary tissue product” comprises one or more fibrous structures, converted or not, that is useful as a wiping implement for post-urinary and post-bowel movement cleaning (toilet tissue), for otorhinolaryngological discharges (facial tissue and/or disposable handkerchiefs), and multi-functional absorbent and cleaning uses (absorbent towels). In one example, a lotion composition-containing multi-ply disposable handkerchief having a caliper of from about 0.1 mm to about 0.4 mm in accordance with the present invention is provided.

“Ply” or “Plies” as used herein means an individual finished fibrous structure optionally to be disposed in a substantially contiguous, face-to-face relationship with other plies, forming a multiple ply finished fibrous structure product and/or sanitary tissue product. It is also contemplated that a single fibrous structure can effectively form two “plies” or multiple “plies”, for example, by being folded on itself.

“Surface of a fibrous structure” as used herein means that portion of the fibrous structure that is exposed to the external environment. In other words, the surface of a fibrous structure is that portion of the fibrous structure that is not completely surrounded by other portions of the fibrous structure.

“User Contacting Surface” as used herein means that portion of the fibrous structure and/or transferable agent and/or surface treating composition and/or lotion composition present directly or indirectly on the surface of the fibrous structure that is exposed to the external environment. In other words, it is that surface formed by the fibrous structure including any surface treating composition and/or lotion composition present directly and/or indirectly on the surface of the fibrous structure that contacts an opposing surface when used by a user. For example, it is that surface formed by the fibrous structure including any surface treating composition and/or lotion composition present directly and/or indirectly on the surface of the fibrous structure that contacts a user's skin when a user wipes his/her skin with the fibrous structure of the present invention.

In one example, the user contacting surface, especially for a textured and/or structured fibrous structure, such as a through-air-dried fibrous structure and/or an embossed fibrous structure, may comprise raised areas and recessed areas of the fibrous structure. In the case of a through-air-dried, pattern densified fibrous structure the raised areas may be knuckles and the recessed areas may be pillows and vice versa. Accordingly, the knuckles may, directly and/or indirectly, comprise the lotion composition and the pillows may comprise the surface treating composition and vice versa so that when a user contacts the user's skin with the fibrous structure, the lotion composition and surface treating composition both contact the user's skin. A similar case is true for embossed fibrous structures with the embossed areas may, directly and/or indirectly, comprise the lotion composition and the non-embossed areas may comprise the surface treating composition and vice versa.

In one example, the user contacting surface has to comprise regions of sufficient size such that two or more different regions (comprising different compositions) are exposed to an opposing surface during use. In other words, a surface of a fibrous structure that is substantially covered (on a microscopic scale) by a lotion composition but completely covered on a macro scale by such lotion composition such that a user's skin is only contacted by the lotion composition does not contain two different regions in its user contacting surface. In one example a user contacting surface may comprise an external layer of a multi-layer fibrous structure wherein the external layer may comprise a surface treating composition and/or a lotion composition.

The user contacting surface may be present on the fibrous structure and/or sanitary tissue product before use by the user and/or the user contacting surface may be created/formed prior to and/or during use of the fibrous structure and/or sanitary tissue product by the user, such as upon the user applying pressure to the fibrous structure and/or sanitary tissue product as the user contacts the user's skin with the fibrous structure and/or sanitary tissue product.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

Fibrous Structure

The transferable agent of the present invention may be present in a surface treating composition and/or a lotion composition and/or may be present, directly or indirectly, on and/or in the fibrous structure.

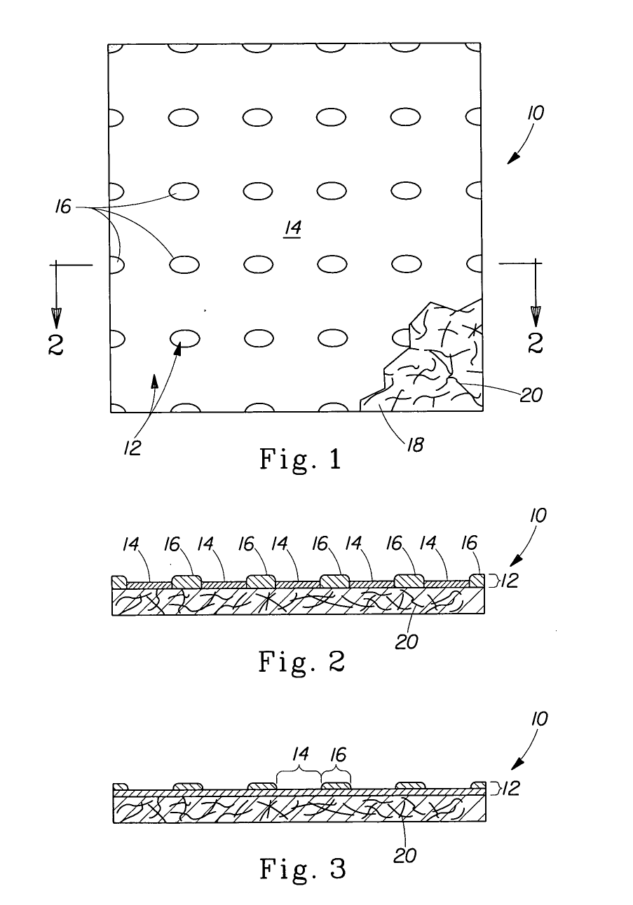

FIG. 1 is a schematic representation of a fibrous structure in accordance with the present invention. As shown in FIG. 1, a fibrous structure 10 comprising a user contacting surface 12 comprising a first region 14 and a second region 16. The user contacting surface 12 is associated with a surface of the fibrous structure 18. As shown, the surface of the fibrous structure 18 may comprise one or more fibers 20.

The first region 14 and/or second region 16 may be present on (associated with) the surface of the fibrous structure 18. When the first region 14 and/or the second region 16 are present on the surface of the fibrous structure 18, one or both may be present on the surface of the fibrous structure 18 in the form of a continuous or substantially continuous network and/or in a plurality of discrete areas (also sometimes known as “islands”).

When present on the surface of the fibrous structure 18, the first region 14 and/or the second region 16 may be in contact with and/or cover the entire or substantially the entire surface area of the surface of the fibrous structure 18. In one example, the first region 18 is in contact with and/or covers the entire or substantially the entire surface area of the surface of the fibrous structure 18.

When present on the surface of the fibrous structure 18, the first region 14 and/or the second region 16 may be in contact with and/or cover less than the entire or substantially the entire surface area of the surface of the fibrous structure 18. In one example, the second region 16 is in contact with and/or covers less than the entire or substantially the entire surface area of the surface of the fibrous structure 18. When either region covers less than substantially the entire surface area of the surface of the fibrous structure 18, that region may be in the form of a plurality of discrete areas.

As shown in FIG. 2, the first region 14 is in contact with and/or covers substantially the entire surface area of the surface of the fibrous structure 18 and the second region 16 is in contact with and/or covers less than substantially the entire surface area of the surface of the fibrous structure 18. The first region 14 may be in the form of a continuous or substantially continuous network and the second region 16 may be in the form of a plurality of discrete areas dispersed throughout the continuous or substantially continuous network of the first region 14.

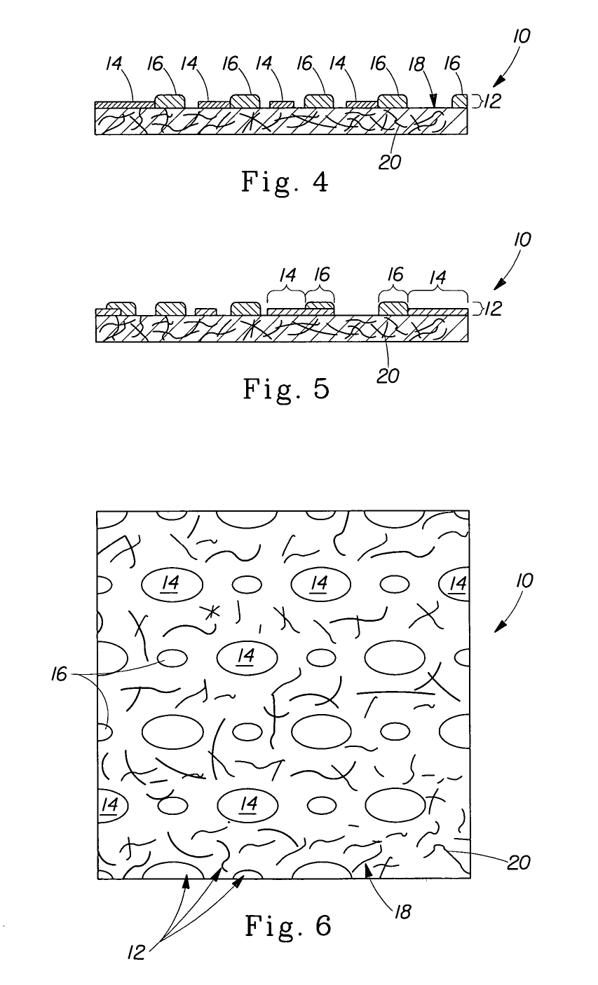

Either region may be in contact with the other region. As shown in FIG. 3, the second region 16 is in contact with the first region 14 such that the first region 14 is positioned between the second region 16 and the surface of the fibrous structure 18. The second region 16 may be present on less than the entire surface area of the first region 14. The second region 16 may be present on the first region 14 in the form of one or more discrete areas. As shown in FIGS. 1, 4 and 5, portions of the second region 16 are in contact with the first region 14 such that both the second region 16 and the first region 14 are in contact directly with the surface of the fibrous structure 18. Also as shown in FIGS. 4 and 5, a portion of the second region 16 is not in contact with the first region 14.

FIGS. 4 and 5 also show that less than substantially the entire surface area of the surface of the fibrous structure 18 is in contacted by or covered by the first region 14 and the second region 16. In these examples, the user contacting surface 12 comprises a third region, namely, the surface of the fibrous structure 18 as well as the first region 14 and the second region 16.

FIG. 6 is a schematic representation of another example of a fibrous structure in accordance with the present invention. The fibrous structure 10 comprises a user contacting surface 12 that comprises a first region 14, a second region 16 and a third region, in this case, the surface of the fibrous structure 18 which comprises one or more fibers 20.

The first region 14 comprises a surface treating composition.

The second region 16 comprises a lotion composition.

In one example, the surface treating composition and/or lotion composition may be present on the surface of the fibrous structure 18 at a greater level by weight than within the fibrous structure.

In another example, the surface treating composition and/or lotion composition may be present within the fibrous structure at a greater level by weight than on the surface of the fibrous structure 18.

The surface area coverage of the surface treating composition on the surface of the fibrous structure may be greater than about 10% and/or greater than about 30% and/or greater than about 50% to about 100% and/or to about 90% and/or to about 85%.

The surface area coverage of the lotion composition on the surface of the fibrous structure may be may be greater than about 1% and/or greater than about 5% and/or greater than about 10% and/or greater than about 20% to about 99% and/or to about 90% and/or to about 75% and/or to about 50%.

In one example, the surface area of the fibrous structure and/or sanitary tissue product comprises greater than about 10% and/or greater than about 20% and/or greater than about 50% and/or greater than about 70% and/or greater than about 80% and/or greater than about 90% of the surface treating composition and from 0 to about 90% and/or from 0 to about 80% and/or from 0 to about 50% and/or from 0 to about 30% and/or from 0 to about 20% and/or from 0 to about 10% of the lotion composition. When the surface area of the surface of the fibrous structure and/or sanitary tissue product comprises 0% of the lotion composition, then the lotion may be within the fibrous structure and/or within the sanitary tissue product, such as between two plies of the sanitary tissue product.

In another example, the surface area of the user contacting surface comprises from about 20% to about 97% and/or from about 50% to about 97% and/or from about 80% to about 97% of the surface treating composition and from about 3% to about 80% and/or from about 3% to about 50% and/or from about 3% to about 20% and/or from about 3% to about 15% of the lotion composition.

Surface area coverage of the fibrous structure and/or sanitary tissue product may be determined by the Surface Area Coverage Test Method described herein.

Each region may, within itself, exhibit differential concentrations of their respective compositions and/or differential elevations (protrusions from the surface of the fibrous structure) of their respective compositions

The user contacting surface area may comprise from greater than about 10% and/or greater than about 30% and/or greater than about 50% to about 100% and/or to about 90% and/or to about 85% of the surface treating composition and/or greater than about 1% and/or greater than about 5% and/or greater than about 10% and/or greater than about 20% to about 99% and/or to about 90% and/or to about 75% and/or to about 50% of the lotion composition.

The combination of the surface treating composition and lotion composition in the user contacting surface exhibits softness greater than a user contacting surface comprising either the surface treating composition or lotion composition alone.

The user contacting surface may be planar or may have protrusions of either the surface treating composition and/or lotion composition such that the user contacting surface exhibits differential elevations.

In another example, the user contacting surface may comprise areas of greater concentration and/or greater elevation of the lotion composition, areas of less concentration and/or lesser elevation of the lotion composition, and areas of the surface treating composition.

The surface treating composition and the lotion composition may comprise one or more similar and/or identical ingredients so long as the user contacting surface comprises a first region comprising a different composition (at least one ingredient differs in the composition) than a composition present in a second region.

Nonlimiting types of fibrous structures according to the present invention include conventionally felt-pressed fibrous structures; pattern densified fibrous structures; and high-bulk, uncompacted fibrous structures. The fibrous structures may be of a homogeneous or multilayered (two or three or more layers) construction; and the sanitary tissue products made therefrom may be of a single-ply or multi-ply construction.

The fibrous structures may be post-processed, such as by embossing and/or calendaring and/or folding and/or printing images thereon.

The fibrous structures may be through-air-dried fibrous structures or conventionally dried fibrous structures.

The fibrous structures may be creped or uncreped.

The fibrous structures and/or sanitary tissue products of the present invention may exhibit a basis weight of between about 10 g/m² to about 120 g/m² and/or from about 12 g/m² to about 80 g/m² and/or from about 14 g/m² to about 65 g/m².

The fibrous structures and/or sanitary tissue products of the present invention may exhibit a total dry tensile strength of greater than about 59 g/cm (150 g/in) and/or from about 78 g/cm (200 g/in) and/or from about 98 g/cm (250 g/in) to about 1182 g/cm (3000 g/in) and/or to about 984 g/cm (2500 g/in) and/or to about 787 g/cm (2000 g/in) and/or to about 394 g/cm (1000 g/in) and/or to about 335 g/cm (850 g/in).

The fibrous structure and/or sanitary tissue products of the present invention may exhibit a density of less than about 0.60 g/cm³ and/or less than about 0.30 g/cm³ and/or less than about 0.20 g/cm³ and/or less than about 0.10 g/cm³ and/or less than about 0.07 g/cm³ and/or less than about 0.05 g/cm³ and/or from about 0.01 g/cm³ to about 0.20 g/cm³ and/or from about 0.02 g/cm³ to about 0.10 g/cm³.

The fibrous structures and/or sanitary tissue products of the present invention may exhibit an average lint value of greater than about 0.1 and/or greater than about 0.5 and/or greater than about 1.0 and/or greater than about 1.5 and/or greater than about 2.0 and/or greater than about 3.0 to about 20 and/or to about 15 and/or to about 13 and/or to about 10 and/or to about 8.

Transferable Agent

The fibrous structure of the present invention may comprise less than about 50% and/or less than about 40% and/or less than about 30% and/or less than about 20% and/or less than about 10% and/or less than about 5% to about 0.01% and/or to about 0.1% and/or to about 1% by weight of the transferable agent.

The transferable agent may be an anti-inflammatory compound, lipid, inorganic anions, inorganic cations, protease inhibitors, sequestration agents and mixtures thereof.

In one example, the transferable agent is associated with the user contacting surface at less than about 10 g/m² and/or less than about 8 g/m² and/or less than about 6 g/m² to about 0.5 g/m² and/or to about 1.0 g/m² and/or to about 1.5 g/m².

In one example, the transferable agent may be any substance that has a higher affinity for oil over water and/or provides a skin health benefit by directly interacting with the skin. Suitable examples of such benefits include, but are not limited to, enhancing skin barrier function, enhancing moisturization and nourishing the skin.

Nonlimiting examples of suitable transferable agents include fatty acids, fatty acid esters, fatty alcohols, triglycerides, phospholipids, mineral oils, essential oils, sterols, sterol esters, emollients, waxes, and combinations thereof.

The transferable agent may be alone, included in a lotion composition and/or included in a surface treating composition. A commercially available lotion composition comprising a transferable agent is Vaseline® Intensive Care Lotion (Chesebrough-Pond's, Inc.).

Nonlimiting examples of fats and oils useful as transferable agents include Apricot Kernel Oil, Avocado Oil, Babassu Oil, Borage Seed Oil, Butter, C.sub.12-C.sub.18 Acid Triglyceride, Camellia Oil, Canola Oil, Caprylic/Capric/Lauric Triglyceride, Caprylic/Capric/Linoleic Triglyceride, Caprylic/Capric/Stearic Triglyceride, Caprylic/Capric Triglyceride, Carrot Oil, Cashew Nut Oil, Castor Oil, Cherry Pit Oil, Chia Oil, Cocoa Butter, Coconut Oil, Cod Liver Oil, Corn Germ Oil, Corn Oil, Cottonseed Oil, C.sub.10-C.sub.18 Triglycerides, Egg Oil, Epoxidized Soybean Oil, Evening Primrose Oil, Glyceryl Triacetyl Hydroxystearate, Glyceryl Triacetyl Ricinoleate, Glycosphingolipids, Grape Seed Oil, Hazelnut Oil, Human Placental Lipids, Hybrid Safflower Oil, Hybrid Sunflower Seed Oil, Hydrogenated Castor Oil, Hydrogenated Castor Oil Laurate, Hydrogenated Coconut Oil, Hydrogenated Cottonseed Oil, Hydrogenated C.sub.12-C.sub.18 Triglycerides, Hydrogenated Fish Oil, Hydrogenated Lard, Hydrogenated Menhaden Oil, Hydrogenated Mink Oil, Hydrogenated Orange Roughy Oil, Hydrogenated Palm Kernel Oil, Hydrogenated Palm Oil, Hydrogenated Peanut Oil, Hydrogenated Shark Liver Oil, Hydrogenated Soybean Oil, Hydrogenated Tallow, Hydrogenated Vegetable Oil, Lanolin and Lanolin Derivatives, Lard, Lauric/Palmitic/Oleic Triglyceride, Lesquerella Oil, Linseed Oil, Macadamia Nut Oil, Maleated Soybean Oil, Meadowfoam Seed Oil, Menhaden Oil, Mink Oil, Moringa Oil, Mortierella Oil, Neatsfoot Oil, Oleic/Linoleic Triglyceride, Oleic/Palmitic/Lauric/Myristic/Linoleic Triglyceride, Oleostearine, Olive Husk Oil, Olive Oil, Omental Lipids, Orange Roughy Oil, Palm Kernel Oil, Palm Oil, Peach Kernel Oil, Peanut Oil, Pengawar Djambi Oil, Pentadesma Butter, Phospholipids, Pistachio Nut Oil, Placental Lipids, Rapeseed Oil, Rice Bran Oil, Safflower Oil, Sesame Oil, Shark Liver Oil, Shea Butter, Soybean Oil, Sphingolipids, Sunflower Seed Oil, Sweet Almond Oil, Tall Oil, Tallow, Tribehenin, Tricaprin, Tricaprylin, Triheptanoin, Trihydroxymethoxystearin, Trihydroxystearin, Triisononanoin, Triisostearin, Trilaurin, Trilinolein, Trilinolenin, Trimyristin, Trioctanoin, Triolein, Tripalmitin, Trisebacin, Tristearin, Triundecanoin, Vegetable Oil, Walnut Oil, Wheat Bran Lipids, Wheat Germ Oil and Zadoary Oil.

Nonlimiting examples of fatty acids suitable as transferable agents include Arachidic Acid, Arachidonic Acid, Behenic Acid, Capric Acid, Caproic Acid, Caprylic Acid, Coconut Acid, Corn Acid, Cottonseed Acid, Hydrogenated Coconut Acid, Hydrogenated Menhaden Acid, Hydrogenated Tallow Acid, Hydroxystearic Acid, Isostearic Acid, Lauric Acid, Linoleic Acid, Linolenic Acid, Linseed Acid, Myristic Acid, Oleic Acid, Palmitic Acid, Palm Kernel Acid, Pelargonic Acid, Ricinoleic Acid, Soy Acid, Stearic Acid, Tall Oil Acid, Tallow Acid, Undecanoic Acid, Undecylenic Acid and Wheat Germ Acid.

Nonlimiting examples of fatty alcohols suitable as transferable agents include Behenyl Alcohol, C.sub.9-C.sub.11 Alcohols, C.sub.12-C.sub.13 Alcohols, C.sub.12-C.sub.15 Alcohols, C.sub.12-C.sub.16 Alcohols, C.sub.14-C.sub.15 Alcohols, Caprylic Alcohol, Cetearyl Alcohol, Cetyl Alcohol, Coconut Alcohol, Decyl Alcohol, Hydrogenated Tallow Alcohol, Lauryl Alcohol, Myristyl Alcohol, Oleyl Alcohol, Palm Alcohol, Palm Kernel Alcohol, Stearyl Alcohol, Tallow Alcohol and Tridecyl Alcohol.

Nonlimiting examples of essential oils suitable as transferable agents include Anise Oil, Balm Mint Oil, Basil Oil, Bee Balm Oil, Bergamot Oil, Birch Oil, Bitter Almond Oil, Bitter Orange Oil, Calendula Oil, California Nutmeg Oil, Caraway Oil, Cardamom Oil, Chamomile Oil, Cinnamon Oil, Clary Oil, Cloveleaf Oil, Clove Oil, Coriander Oil, Cypress Oil, Eucalyptus Oil, Fennel Oil, Gardenia Oil, Geranium Oil, Ginger Oil, Grapefruit Oil, Hops Oil, Hyptis Oil, Indigo Bush Oil, Jasmine Oil, Juniper Oil, Kiwi Oil, Laurel Oil, Lavender Oil, Lemongrass Oil, Lemon Oil, Linden Oil, Lovage Oil, Mandarin Orange Oil, Matricaria Oil, Musk Rose Oil, Nutmeg Oil, Olibanum, Orange Flower Oil, Orange Oil, Patchouli Oil, Pennyroyal Oil, Peppermint Oil, Pine Oil, Pine Tar Oil, Rose Hips Oil, Rosemary Oil, Rose Oil, Rue Oil, Sage Oil, Sambucus Oil, Sandalwood Oil, Sassafras Oil, Silver Fir Oil, Spearmint Oil, Sweet Marjoram Oil, Sweet Violet Oil, Tar Oil, Tea Tree Oil, Thyme Oil, Wild Mint Oil, Yarrow Oil and Ylang Ylang Oil.

Nonlimiting examples of sterols and/or sterol derivatives suitable as transferable agents include sterols having a tail on the 17 position and having no polar groups for example cholesterol, sitosterol, stigmasterol, and ergosterol, as well as, C₁₀-C₃₀ cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, avocadin and sterol esters.

Nonlimiting examples of emollients suitable as transferable agents include Mineral Oil, Mineral Jelly, Petrolatum, cosmetic esters, fatty esters, glyceryl esters, alkoxylated carboxylic acids, alkoxylated alcohols, fatty alcohols, lanolin and lanolin derivatives, petrolatum base oils, silicones, fats, hydrogenated vegetable oils and polyhydroxy esters.

Nonlimiting examples of waxes suitable as transferable agents include natural and synthetic waxes, such as bayberry wax, beeswax, C₃₀ alkyl dimethicone, candelilla wax, carnuaba, ceresin, cetyl esters, hydrogenated cottonseed oil, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, motan acid wax, motan wax, ouricury wax, ozokerite, paraffin, PEG-6 beeswax, PEG-8 beeswax, rice bran wax, shellac wax, spent grain wax, steryl dimethicone synthetic beeswax, synthetic candelilla wax, synthetic carnuba wax, synthetic japan wax, synthetic jojoba wax and synthetic wax. In one example, the wax comprises carnuba, cerasin, cetyl esters, microcrystalline wax, montan wax, ozokerite and/or synthetic wax.

Nonlimiting examples of humectants suitable as transferable agents include Acetamide MEA, Aloe Vera Gel, Arginine PCA, Chitosan PCA, Copper PCA, Corn Glycerides, Dimethyl Imidazolidinone, Fructose, Glucamine, Glucose, Glucose Glutamate, Glucuronic Acid, Glutamic Acid, Glycereth-7, Glycereth-12, Glycereth-20, Glycereth-26, Glycerin, Honey, Hydrogenated Honey, Hydrogenated Starch Hydrolysate, Hydrolyzed Corn Starch, Lactamide MEA, Lactic Acid, Lactose Lysine PCA, Mannitol, Methyl Gluceth-10, Methyl Gluceth-20, PCA, PEG-2 Lactamide, PEG-10 Propylene Glycol, Polyamino Sugar Condensate, Potassium PCA, Propylene Glycol, Propylene Glycol Citrate, Saccharide Hydrolysate, Saccharide Isomerate, Sodium Aspartate, Sodium Lactate, Sodium PCA, Sorbitol and TEA-Lactate.

Surface Treating Composition

A surface treating composition, for purposes of the present invention, is a composition that improves the tactile sensation of a surface of a fibrous structure perceived by a user whom holds a fibrous structure and/or sanitary tissue product comprising the fibrous structure and rubs it across the user's skin. Such tactile perceivable softness can be characterized by, but is not limited to, friction, flexibility, and smoothness, as well as subjective descriptors, such as a feeling like lubricious, velvet, silk or flannel.

The surface treating composition may or may not be transferable. Typically, it is substantially non-transferable.

The surface treating composition may increase or decrease the surface friction of the surface of the fibrous structure, especially the user contacting surface of the fibrous structure. Typically, the surface treating composition will reduce the surface friction of the surface of the fibrous structure compared to a surface of the fibrous structure without such surface treating composition.

The surface treating composition may have a wettability tension less than or equal to the surface tension of the lotion composition so as to minimize the spreading of the lotion composition that comes into contact with the surface treating composition.

The surface treating composition comprises a surface treating agent. The surface treating composition during application to the fibrous structure may comprise at least about 0.1% and/or at least 0.5% and/or at least about 1% and/or at least about 3% and/or at least about 5% to about 90% and/or to about 80% and/or to about 70% and/or to about 50% and/or to about 40% by weight of the surface treating agent. In one example, the surface treating composition comprises from about 5% to about 40% by weight of the surface treating agent.

The surface treating composition present on the fibrous structure and/or sanitary tissue product comprising the fibrous structure of the present invention may comprise at least about 0.01% and/or at least about 0.05% and/or at least about 0.1% of total basis weight of the surface treating agent. In one example, the fibrous structure and/or sanitary tissue product may comprise from about 0.01% to about 20% and/or from about 0.05% to about 15% and/or from about 0.1% to about 10% and/or from about 0.01% to about 5% and/or from about 0.1% to about 2% of total basis weight of the surface treating composition.

In one example, the surface treating composition of the present invention is a microemulsion of a surface treating agent (for example an aminofunctional polydimethylsiloxane) in water. In such an example, the concentration of the surface treating agent within the surface treating composition may be from about 3% to about 60% and/or from about 4% to about 50% and/or from about 5% to about 40%. Nonlimiting examples of such microemulsions are commercially available from Wacker Chemie, Dow Corning and/or General Electric Silicones.

Nonlimiting examples of suitable surface treating agents can be selected from the group consisting of: polymers such as polyethylene and derivatives thereof, hydrocarbons, waxes, oils, silicones (polysiloxanes), quaternary ammonium compounds, fluorocarbons, substituted C₁₀-C₂₂ alkanes, substituted C₁₀-C₂₂ alkenes, in particular derivatives of fatty alcohols and fatty acids(such as fatty acid amides, fatty acid condensates and fatty alcohol condensates), polyols, derivatives of polyols (such as esters and ethers), sugar derivatives (such as ethers and esters), polyglycols (such as polyethyleneglycol) and mixtures thereof.

The surface treating composition may comprise additional ingredients such as a vehicle as described herein below which may not be present on the fibrous structure and/or sanitary tissue product comprising such fibrous structure. In one example, the surface treating composition may comprise a surface treating agent and a vehicle such as water to facilitate the application of the surface treating agent onto the surface of the fibrous structure.

Lotion Composition

The lotion composition may comprise oils and/or emollients and/or waxes (any and all of which may be a transferable agent) and/or immobilizing agents. In one example, the lotion composition comprises from about 10% to about 90% of an oil and/or liquid emollient and from about 10% to about 50% of immobilizing agent and/or from about 0% to about 60% of petrolatum and optionally the balance of a vehicle.

The lotion compositions may be heterogeneous. They may contain solids, gel structures, polymeric material, a multiplicity of phases (such as oily and water phase) and/or emulsified components. It may be difficult to determine precisely the melting temperature of the lotion composition, i.e. difficult to determine the temperature of transition between the liquid form, the quasi-liquid from, the quasi-solid form and the solid form. The terms melting temperature, melting point, transition point and transition temperature are used interchangeably in this document and have the same meaning.

The lotion compositions may be semi-solid, of high viscosity so they do not substantially flow without activation during the life of the product or gel structures.

The lotion compositions may be shear thinning and/or they may strongly change their viscosity around skin temperature to allow for transfer and easy spreading on a user's skin.

The lotion compositions may be in the form of emulsions and/or dispersions.

In one example of a lotion composition, the lotion composition has a water content of less than about 20% and/or less than 10% and/or less than about 5% or less than about 0.5%.

In another example, the lotion composition may have a solids content of at least about 15% and/or at least about 25% and/or at least about 30% and/or at least about 40% to about 100% and/or to about 95% and/or to about 90% and/or to about 80%.

Nonlimiting examples of suitable oils and/or emollients include glycols (such as propylene glycol and/or glycerine), polyglycols (such as triethylene glycol), petrolatum, fatty acids, fatty alcohols, fatty alcohol ethoxylates, fatty alcohol esters and fatty alcohol ethers, fatty acid ethoxylates, fatty acid amides and fatty acid esters, hydrocarbon oils (such as mineral oil), squalane, fluorinated emollients, silicone oil (such as dimethicone) and mixtures thereof.

Immobilizing agents include agents that are may prevent migration of the emollient into the fibrous structure such that the emollient remain primarily on the surface of the fibrous structure and/or sanitary tissue product and/or on the surface treating composition on a surface of the fibrous structure and/or sanitary tissue product and facilitate transfer of the lotion composition to a user's skin. Immobilizing agents may function as viscosity increasing agents and/or gelling agents.

Nonlimiting examples of suitable immobilizing agents include waxes (such as ceresin wax, ozokerite, microcrystalline wax, petroleum waxes, fisher tropsh waxes, silicone waxes, paraffin waxes), fatty alcohols (such as cetyl and/or stearyl alcohol), fatty acids and their salts (such as metal salts of stearic acid), mono and polyhydroxy fatty acid esters, mono and polyhydroxy fatty acid amides, silica and silica derivatives, gelling agents, thickeners and mixtures thereof.

In one example, the lotion composition comprises at least one immobilizing agent and at least one emollient.

In one example, the lotion composition comprises a sucrose ester of a fatty acid.

The lotion composition may be added to a fibrous structure at any point during the papermaking and/or converting process. In one example, the lotion composition is added to the fibrous structure during the converting process.

The lotion composition may be comprise a transferable agent and thus be considered a transferable lotion composition. A transferable lotion composition comprises at least one transferable agent that is capable of being transferred to an opposing surface such as a user's skin upon use. In one example, at least 0.1% of the transferable lotion present on the user contacting surface transfers to the user's skin during use. The amount of transferable composition that transfers to a user's skin during use can be determined by known methods such as by tape stripping the skin 3 times, after use of the fibrous structure and/or sanitary tissue product by the user, with Tegaderm Tapes, available from 3M, and analyzing the tapes for the transferable composition or a component within the transferable composition assuming all components of the transferable composition transfer equally.

Other optional components that may be included in the lotion composition include vehicles, perfumes, especially long lasting and/or enduring perfumes, antibacterial actives, antiviral actives, disinfectants, pharmaceutical actives, film formers, deodorants, opacifiers, astringents, solvents, cooling sensate agents, and the like. Particular examples of lotion composition components include camphor, thymol, menthol, chamomile extracts, aloe vera, calendula officinalis, alpha bisalbolol, Vitamin E, Vitamin E acetate.

In one example of the lotion composition of the present invention, the lotion composition has a melting point greater than about 35° C. For example, the lotion composition has to be subjected to a temperature of greater than about 35° C. before a substantial amount (for example, greater than 30% and/or greater than 40% and/or greater than 50% and/or greater than 60%) of the lotion composition melts. This can be expressed as:
(1) ΔH²/ΔH¹ is equal to or larger than about 1 and/or equal to or larger than about 4 and/or equal to or larger than about 9; and/or
(2) ΔH² is equal to or larger than about 30 J/g, 40 J/g and/or equal to or larger than about 60 J/g (especially if ΔH¹ is 0)
   wherein: ΔH¹ is the energy required to raise the temperature of the lotion composition from 15° C. to 35° C.; ΔH² is the energy required to raise the temperature of the lotion composition from 35° C. to the temperature where the lotion composition is fully liquid or where no more melting occurs below 100° C. in the case the lotion composition contains components only melting above 100° C.

ΔH is measured by DSC technique using standard parameters known to the one skilled in the art. DSC data are obtained using a Thwing Albert DSC 2920 Instrument, calibrated with an indium metal standard with a melting onset temperature of 156.6° C. and a heat of melting of 6.80 calories per gram, as reported in the literature. The sample is first heated to 100° C. at a rate of 10° C./min, equilibrated for 5 minutes at 100° C., cooled down to −30° C. at a rate of −2.5° C./min, equilibrated at −30° C. for 5 minutes and then finally heated from −30° C. to +100° C. at a rate of 2.5° C./min to evaluate the melt behaviour. For determination of ΔH¹ and ΔH² the final heating ramp is used. ΔH¹ is the area between the DSC curve and the baseline between 15° C. and 35° C. and ΔH² is the area between the DSC curve and the baseline between 35° C. and the temperature where the lotion composition is fully liquid or where no more melting occurs below 100° C. in the case the lotion composition contains components only melting above 100° C. By way of example, a lotion composition of the present invention that comprises about 40% Stearylalcohol, about 30% Mineral oil and about 30% Petrolatum has a value of ΔH²/ΔH¹>9 and a value of ΔH²>60 J/g.

In one example, the lotion composition is present on the surface of the fibrous structure and/or sanitary tissue product and/or on the surface treating composition present on the surface of the fibrous structure and/or sanitary tissue product at a level of at least about 0.5 g/m² and/or at least about 1.0 g/m² and/or at least about 1.5 g/m² per user contacting surface. In another example, the lotion composition is present on the surface of the fibrous structure and/or sanitary tissue product and/or on the surface treating composition present on the surface of the fibrous structure and/or sanitary tissue product at a level of from about 0.5 g/m² and/or from about 1.0 g/m² and/or from about 1.5 g/m² to about 10 g/m² and/or to about 8 g/m² and/or to about 6 g/m² per user contacting surface.

Vehicle

As used herein a “vehicle” is a material that can be used to dilute and/or emulsify agents forming the surface treating composition and/or lotion composition to form a dispersion/emulsion. A vehicle may be present in the surface treating composition and/or lotion composition, especially during application of the surface treating composition and/or to the fibrous structure. A vehicle may dissolve a component (true solution or micellar solution) or a component may be dispersed throughout the vehicle (dispersion or emulsion). The vehicle of a suspension or emulsion is typically the continuous phase thereof. That is, other components of the dispersion or emulsion are dispersed on a molecular level or as discrete particles throughout the vehicle.

Suitable materials for use as the vehicle of the present invention include hydroxyl functional liquids, including but not limited to water. In one example, the lotion composition comprises less than about 20% and/or less than about 10% and/or less than about 5% and/or less than about 0.5% w/w of a vehicle, such as water. In one example, the surface treating composition comprises greater than about 50% and/or greater than about 70% and/or greater than about 85% and/or greater than about 95% and/or greater than about 98% w/w of a vehicle, such as water.

Process Aids

Process aids may also be used in the lotion compositions of the present invention. Nonlimiting examples of suitable process aids include brighteners, such as TINOPAL CBS-X®, obtainable from CIBA-GEIGY of Greensboro, N.C.

### NONLIMITING EXAMPLES OF LOTION COMPOSITIONS

### Example 1 of Lotion Composition

| | |
|---|---|
| Stearyl Alcohol CO1897* | 40% w/w |
| Petrolatum Snowwhite V28EP** | 30% w/w |
| Mineral oil Carnation** | 30% w/w |

*Available from Procter&Gamble Chemicals, Cincinnati, USA
**Available from Crompton Corporation

The lotion composition has a melting point of about 51° C. and a melt viscosity at 56° C. of about 17 m*Pas measured at a shear rate of 0.1 l/s. The mineral oil used in this formulation has a viscosity of about 21 mPa*s at 20° C. The lotion composition can be applied to one or both surfaces of the fibrous structure at total add-on levels of 3.6 g/m², 4.2 g/m², 6 g/m², 7.2 g/m², 8.4 g/m² and 11.4 g/m².

Processes for Treating Fibrous Structures and/or Sanitary Tissue Products

a. Transferable Agent and/or Surface Treating Composition:

Any contact or contact free application suitable for applying the transferable agent and/or surface treating composition, such as spraying, dipping, padding, printing, slot extruding, rotogravure printing, flexographic printing, offset printing, screen printing, mask or stencil application process and mixtures thereof can be used to apply the transferable agent and/or surface treating composition to the fibrous structure and/or sanitary tissue product and/or to the lotion composition present on a surface of the fibrous structure and/or sanitary tissue product. The transferable agent and/or surface treating composition can be applied to the fibrous structure and/or sanitary tissue product before, concurrently, or after the lotion composition application to the fibrous structure and/or sanitary tissue product. The transferable agent and/or surface treating composition can be applied during papermaking and/or converting, especially if applied to the outside layer of a layered fibrous structure and/or sanitary tissue product comprising such layered fibrous structure.

In one example, the surface treating composition is applied by an application process that provides a relatively high surface area coverage on the surface of the fibrous structure and/or sanitary tissue product. Examples of such suitable application process include, but are not limited to, printing, slot extruding and/or spraying with fine particles (although spraying has disadvantage of producing aerosoles if high area coverage is to be achieved).

b. Transferable Agent and/or Lotion Composition:

Any contact or contact free application suitable for applying the transferable agent and/or lotion composition, such as spraying, dipping, padding, printing, slot extruding, rotogravure printing, flexographic printing, offset printing, screen printing, mask or stencil application process and mixtures thereof can be used to apply the transferable agent and/or lotion composition to the fibrous structure and/or sanitary tissue product and/or surface treating composition present on the surface of the fibrous structure and/or sanitary tissue product. The transferable agent and/or lotion composition can be applied to the fibrous structure and/or sanitary tissue product before, concurrently, and/or after the surface treating composition application to the fibrous structure and/or sanitary tissue product. In one example, the transferable agent and/or lotion composition is applied to the surface treating composition present on the surface of the fibrous structure and/or sanitary tissue product.

In one example, the transferable agent and/or lotion composition is applied by an application process that provides a relatively low surface area coverage on the surface of the fibrous structure and/or sanitary tissue product and/or on the surface treating composition present on the surface of the fibrous structure and/or sanitary tissue product such that regions of surface treating composition and regions of lotion composition produce the user contacting surface. Example of such suitable application processes include, but are not limited to, spraying, especially spraying with rotating discs, printing, slot extruding in stripes and/or other patterns.

In one example, the surface treating composition may be added to a fiber furnish that will form an external layer of a multilayer fibrous structure. The transferable agent and/or lotion composition may be applied to the surface formed by the external layer of the multilayer fibrous structure.

In one example, the surface treating composition is applied to the surface of the fibrous structure during the fibrous structure making process, such as before and/or after drying the fibrous structure. The transferable agent and/or lotion composition may then be applied to the surface treating composition on the surface of the fibrous structure during the converting process.

In one example, the surface treating composition contains less than about 5% and/or less than about 3% and/or less than about 1% and/or less than about 0.5% moisture at the time the lotion composition is applied to it.

Applying Transferable Agent and/or Lotion Composition to Fibrous Structures:

In one example, directly following the application of a surface treating composition to a surface of a fibrous structure, the transferable agent and/or lotion composition is applied to the fibrous structure and/or sanitary tissue product. The fibrous structure and/or sanitary tissue product span between the two operations was about 5 meter. A commercially available rotary spray application system RFT-Compact-III with applicator heads for the tissue and textile industry (available from Weitmann&Konrad GmbH & Co KG, Leinfelden Echterdingen, Germany) was modified to be used to practice the present invention. The application head is equipped with 5 sets of rotary disks (type 1/1) and has an effective application width of 448 mm. The housing of the application head was replaced with water heated walls on the top, the bottom and the rear side of the application head. The whole unit was then insulated towards the outside. Two of these modified application heads were used, installed facing each other so that both sides of a fibrous structure and/or sanitary tissue product can be treated simultaneously. Heating units with an integrated pump (Type W60/10-12/40, available from Kelviplast GmbH, Germany) are used to supply the application units with water of the desired temperature. In particular, the design of the heating elements was chosen so that the temperature inside the application head is within +/−2° C. from the target temperature. The transferable agent and/or lotion composition infeed of the application heads are connected through a heat traced piping system to a heated pump that is connected through heat traced piping to a heated 100 liter tank that holds the melted transferable agent and/or lotion composition. The return lines of the applicator feed back into the heated tank. A heated flow meter was installed in the lotion composition supply line between pump and application heads. The flow meter (Promass 63M, available from Endress & Hauser, Switzerland) was connected to the control unit of the RFT-Compact-III system that was then used to control the transferable agent and/or lotion composition pump (Gear pump of type Labu Brox) to deliver the desired transferable agent and/or lotion composition flow to the application heads.

No changes are made to the setup, shape and dimensions of the rotating surfaces in the commercially available application head. Each set of rotating surfaces consisted of 2 rotating discs stacked on top of each other. The transferable agent and/or lotion composition supply to the two rotating surfaces of each stack is equally split. The discs have a diameter of about 98 mm. The five individual stacks of rotating surfaces are spaced apart by about 112 mm. The first, third and fifth set of rotating surfaces is installed vertically shifted versus the second and fourth stack of rotating surfaces to avoid interference between the horizontally overlapping streams of droplets. The sets of rotating surfaces are commercially available from Weitmann & Konrad GmbH & Co, Germany (type 1/1, Art. No. 618996 [upper set] and 618997 [lower set]). The applicator is operated horizontally and with a distance of about 154 mm between the fibrous structure and/or sanitary tissue product and the center of the disks. The fibrous structure and/or sanitary tissue product is run vertically from top to bottom between the two application heads. Controlled by the windows in the housing between the rotating surfaces and the fibrous structure and/or sanitary tissue product, each stack of rotating surfaces covers a cross direction width of about 224 mm on the fibrous structure and/or sanitary tissue product with the exception of the two outer stacks of rotating surfaces of the applicator which only cover 112 mm each. At each position the streams of two stacks of rotating surfaces are overlapping. Even distribution to the individual stacks of discs was achieved with throttles of 1 mm diameter, installed between the infeeds to the rotary discs and the central supply pipe of the applicator. The transferable agent and/or lotion composition temperature is controlled to a determined value through the heating of the tank, the piping and the temperature in the application heads to the desired value. The flow rate is adjusted to achieve the desired add-on level of the fibrous structure. During application, the fibrous structure and/or sanitary tissue product is typically kept at room temperature. The transferable agent and/or lotion composition almost instantaneously solidifies after impacting the fibrous structure and/or sanitary tissue product.

### TEST METHODS

A. Transferable Agent Transfer Test Method

The transfer amount and/or efficiency of the transferable agent to an opposing surface can be measured by the following method.

First, determine the amount of lotion present in and/or on the surface of a fibrous structure. Methods for determining such an amount are known in the art, such as PNMR and near IR spectroscopy. An example of a method is described in U.S. Pat. No. 6,261,580 the description of which is, in relevant part, incorporated herein by reference.

Next, the amount of the lotion that transfers to an opposing surface is determined. Again, methods for doing so are known in the art. U.S. Pat. No. 6,261,580 describes and example of such a method.

The amount of transferable agent transferred from a treated tissue product is determined with a Sutherland Rub Tester (available from Testing Machines, Inc. of Amityville, N.Y.). This tester uses a motor to rub a sample of the treated tissue 5 times over an impervious transfer surface. Any transferable agent transferred from the treated tissue is extracted from the transfer surface and the quantity transferred is determined using gas chromatographic methods.

Prior to the transferable agent transfer testing, the fibrous structure samples to be tested should be conditioned according to TAPPI Method #T402OM-88. Here, samples are preconditioned for 24 hours at a relative humidity level of 10 to 35% and within a temperature range of 22 to 40° C. After this preconditioning step, samples should be conditioned for 24 hours at a relative humidity of 48 to 52% and within a temperature range of 22 to 24° C. Transfer testing should also take place within the confines of the constant temperature and humidity room.

Next, obtain a 30″ (76 cm)×40″ (101 cm) piece of Crescent #300 cardboard from Cordage Inc. of Cincinnati, Ohio. Using a paper cutter, cut out six pieces of cardboard of dimensions of 2.25″×7.25″ (5.7 cm×18.4 cm). Draw two lines parallel to the short dimension and down 1.125″ (2.9 cm) from the top and bottom most edges on the white side of the cardboard. Carefully score the length of the line with a razor blade using a straight edge as a guide. Score it to a depth about half way through the thickness of the sheet. This scoring allows the cardboard/felt combination to fit tightly around the weight of the Sutherland Rub tester. Draw an arrow running parallel to the long dimension of the cardboard on this scored side of the cardboard.

Then, cut the six pieces of black felt (F-55 or equivalent from New England Gasket of Bristol, Conn.) to the dimensions of 2.25″×8.5″×0.0625″ (5.7 cm×21.6 cm×1.6 cm). Place the felt on top of the unscored, green side of the cardboard such that the long edges of both the felt and cardboard are parallel and in alignment. Make sure the fluffy side of the felt is facing up. Also allow about 0.5″ (1.3 cm) to overhang the top and bottom most edges of the cardboard. Cut a fibrous structure sample to the same dimensions as the felt and center it on the felt. Snugly fold the overhanging edges and tape (Scotch.TM. tape from 3M, St. Paul, Minn. is suitable) the sample and the felt to the back of the cardboard to complete preparation of a felt/cardboard/sample.

A 4 pound (1.8 kilogram) weight has 4 cm² (26 cm²) of effective contact area providing a contact pressure of 1 psi (6.8 kPa). Since the contact pressure can be changed by alteration of the rubber pads mounted on the face of the weight, it is important to use only the rubber pads supplied by the manufacturer (Brown Inc., Mechanical Services Department, Kalamazoo, Mich.). These pads must be replaced if they become hard, abraded or chipped off.

When not in use, the weight must be positioned such that the pads are not supporting the full weight of the weight. It is best to store the weight on its side.

For the measurement of the actual tissue paper/cardboard combinations, place the transfer surface (glass mirror) on the base plate of the tester positioning the mirror against the hold-down pins. The hold-down pins prevent the mirror from moving during the test.

Clip the calibration felt/cardboard/sample onto the four pound weight with the cardboard side contacting the pads of the weight. Make sure the cardboard/felt/tissue combination is resting flat against the weight. Hook this weight onto the tester arm. The felt/cardboard/tissue sample must rest flat on the mirror and must be in 100% contact with the mirror surface.

Next, activate the tester by depressing the “push” button. At the end of the five strokes the tester will automatically stop.

Remove the weight with the felt covered cardboard. Inspect the tissue sample. If torn, discard the felt and tissue and start over. If the tissue sample is intact, remove the felt covered cardboard from the weight. Repeat with an additional three felt/cardboard/tissue samples to insure sufficient lotion has been transferred for accurate measurement.

Repeat the above steps to generate six replicates for each test condition.

After all conditions have been measured, remove and discard all felt. Felts strips are not used again. Cardboard supports are used until they are bent, torn, limp, or no longer have a smooth surface.

Each mirror is washed once with a four milliliter aliquot of toluene into a beaker. The extract is transferred to a sample vial and dried down using dry nitrogen. The mirror is washed a second time with a two milliliter aliquot of toluene, the liquid transferred and dried as described above.

One milliliter of toluene is then added to each sample vial before sealing the vial. The vials are then gently agitated to dissolve the transferred mirror extract. The level of transferable agent in the dissolved extract is then measured using known gas chromatographic techniques.

Known standards are used, as is common in the art, to determine transferable agent recovery constants (for the washing and transfer steps) and to determine gas chromatograph equipment constants.

The amount of transferable agent chromatographically determined is divided by recovery constant to estimate the amount of transferable agent on the mirror. Results are reported in milligrams.

B. Releative Concentration of Composition on Surface Test Method

Relative concentration of a composition on a surface of the fibrous structure and/or sanitary tissue product may be determined by using near IR spectroscopy, especially if the sample contains a hydrocarbon-containing composition. The near IR spectroscopy method may use a filter photomer or other near IR instrument, but it must be configured for back scatter detection. Appropriate wavelengths are used.

The fibrous structure and/or sanitary tissue product is placed under the near IR instrument and a reading is obtained. The sample is then turned over to obtain a reading from the other side of the sample.

In addition to near IR, mid-IR spectroscopy with suitable equipment and wavelengths may also be used to determine relative concentration of a composition on a surface of a fibrous structure and/or sanitary tissue product.

All documents cited in the Detailed Description of the Invention are, in relevant part, incorporated herein by reference, the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A fibrous structure comprising a transferable agent, wherein the transferable agent is associated with the fibrous structure such that greater than about 6% by weight of the transferable agent is transferred from the fibrous structure to a surface during use by a user.

2. The fibrous structure according to claim 1 wherein the fibrous structure comprises less than about 50% by weight of the fibrous structure of the transferable agent.

3. The fibrous structure according to claim 1 wherein the transferable agent is associated with a user contacting surface present on a surface of the fibrous structure.

4. The fibrous structure according to claim 3 wherein the transferable agent is associated with the user contacting surface at less than about 10 g/m².

5. The fibrous structure according to claim 1 wherein the transferable agent is present in a lotion composition further comprising a compound selected from the group consisting of: hydrocarbons, fatty acid esters, alcohol ethoxylates and mixtures thereof.

6. The fibrous structure according to claim 5 wherein the lotion composition further comprises a softening agent.

7. The fibrous structure according to claim 1 wherein the transferable agent comprises a skin care agent.

8. The fibrous structure according to claim 1 wherein the transferable agent is present at a higher concentration on a surface of the fibrous structure compared to within the fibrous structure.

9. The fibrous structure according to claim 8 wherein the transferable agent covers less than the entire surface area of the surface of the fibrous structure.

10. The fibrous structure according to claim 9 wherein the surface of the fibrous structure further comprises a surface treating composition.

11. The fibrous structure according to claim 10 wherein the surface treating composition comprises a surface treating agent selected from the group consisting of: polymers, hydrocarbons, waxes, oils, silicones, quaternary ammonium compounds, fluorocarbons, substituted C₁₀-C₂₂ alkanes, substituted C₁₀-C₂₂ alkenes, polyols, sugar derivatives and mixtures thereof.

13. The fibrous structure according to claim 10 wherein the fibrous structure exhibits a softness value that is greater than the softness value of the fibrous structure without the transferable agent.

14. The fibrous structure according to claim 1 wherein the transferable agent comprises a medicinal agent.

15. The fibrous structure according to claim 14 wherein a clinically beneficial amount of the medicinal agent is transferable from the fibrous structure to a user's skin.

16. A single- or multi-ply sanitary tissue product comprising a fibrous structure according to claim 1.

17. A process for treating a fibrous structure with a transferable agent, the process comprising the step of associating a transferable agent to a fibrous structure in need of treatment such that greater than about 6% by weight of the transferable agent is transferred from the fibrous structure to a surface during use by a user.
